(19) United States
(12) Patent Application Publication
(10) Pub. No.: US 2008/0161641 A1
(43) Pub. Date: Jul. 3, 2008

(54) PROCEDURE

(75) Inventors: Takeharu NAKAZATO, Tokyo (JP); Tetsuya YAMAMOTO, Hanno-shi (JP); Takumi DEJIMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/965,199

(22) Filed: Dec. 27, 2007

(30) Foreign Application Priority Data
Dec. 28, 2006 (JP) 2006-356336

(51) Int. Cl. A61B 1/018

(52) U.S. Cl. 600/104

(57) ABSTRACT

A procedure for forming an opening in a luminal organ surface while observing images from an endoscope introduced into a luminal organ through a natural orifice, and performing observation and treatment of an organ in an abdominal cavity through the opening, including the steps of: inserting an endoscope into a target luminal organ through a natural orifice of a subject; checking and identifying a region without other organs or blood vessels under observation by the endoscope; inserting a perforating treatment instrument for perforating the target luminal organ under observation by the endoscope; perforating the region where safety in an abdominal cavity outside the target luminal organ is checked in the checking step, using the perforating treatment instrument; inserting the endoscope into the abdominal cavity through the perforated portion; inserting a surgical treatment instrument for performing treatment of the organ in the abdominal cavity through the perforated portion; performing surgery under observation by the endoscope; removing the endoscope and the surgical treatment instrument from the perforated portion; inserting a suturing instrument for suturing the perforated portion under observation by the endoscope; and suturing the perforated portion using the suturing instrument under observation by the endoscope, wherein the endoscope is replaced by an ultrasonic endoscope in at least one of the steps.

### CROSS REFERENCE TO RELATED APPLICATION

This application claims benefit of Japanese Application No. 2006-356336 filled in Japan on Dec. 28, 2006, the contents of which are incorporated by this reference.

### BACKGROUND OF THE INVENTION

1. Field of the Invention

The present invention relates to a procedure for forming an opening in a luminal organ surface while observing images from an endoscope introduced into a luminal organ through a natural orifice, and performing observation and treatment of an organ in an abdominal cavity through the opening.

2. Description of the Related Art

Researches have been advanced on surgery referred to as a NOTES (Natural Orifice Translumenal Endoscopic Surgery) procedure for forming an opening in a luminal organ wall surface while observing images from an endoscope introduced into a luminal organ through a natural orifice, and introducing the endoscope through the opening into an abdominal cavity to perform observation and treatment of an organ in the abdominal cavity, and various proposals on endoscopes and treatment instrument systems used therefore have been made as disclosed in, for example, Japanese Patent Laid-Open No. 2004-267772.

### SUMMARY OF THE INVENTION

The present invention provides a procedure including the steps of inserting an endoscope into a target luminal organ through a natural orifice of a subject; checking and identifying a region without other organs or blood vessels under observation by the endoscope; inserting a perforating treatment instrument for perforating the target luminal organ under observation by the endoscope; perforating the region where safety in an abdominal cavity outside the target luminal organ is checked by the checking step, using the perforating treatment instrument; inserting the endoscope into the abdominal cavity through the perforated portion; inserting a surgical treatment instrument for performing treatment of an organ in the abdominal cavity through the perforated portion; performing surgery under observation by the endoscope; removing the endoscope and the surgical treatment instrument from the perforated portion; inserting a suturing instrument for suturing the perforated portion under observation by the endoscope; and suturing the perforated portion using the suturing instrument under observation by the endoscope, wherein the endoscope is replaced by an ultrasonic endoscope in at least one of the steps.

The above and other objects, features and advantages of the invention will become more clearly understood from the following description referring to the accompanying drawings.

### BRIEF DESCRIPTION OF THE DRAWINGS

FIG. 1 is a schematic block diagram of the entire configuration of an ultrasonic endoscope system;

FIG. 2 is a schematic block diagram of an example of a perforating treatment instrument in the ultrasonic endoscope system;

FIG. 3 is a schematic block diagram of another example of a perforating treatment instrument in the ultrasonic endoscope system;

FIG. 4 illustrates an operation of the ultrasonic endoscope system, and shows the steps of inserting an ultrasonic endoscope into a body cavity and observing inside and outside a stomach;

FIG. 5 illustrates an operation of the ultrasonic endoscope system, and shows a section taken along the line [V]-[V] in FIG. 4 in an enlarged manner;

FIG. 6 illustrates an operation of the ultrasonic endoscope system, and shows the steps of inserting the perforating treatment instrument into the stomach through a treatment instrument channel in the ultrasonic endoscope and perforating a predetermined region;

FIG. 7 illustrates an operation of the ultrasonic endoscope system, and shows a section taken along the line [VII]-[VII] in FIG. 6 in an enlarged manner;

FIG. 8 illustrates an operation of the ultrasonic endoscope system, and shows the steps of perforating the predetermined region using the perforating treatment instrument and performing insufflation;

FIG. 9 illustrates an operation of the ultrasonic endoscope system, and shows the steps of inserting the ultrasonic endoscope through the perforated portion and observing a target region;

FIG. 10 illustrates an operation of the ultrasonic endoscope system, and shows the steps of inserting the ultrasonic endoscope and a surgical treatment instrument through the perforated portion into an abdominal cavity and performing surgery;

FIG. 11 illustrates an operation of the ultrasonic endoscope system, and is an enlarged view of essential portions showing the steps of inserting a suturing instrument and suturing the perforated portion; and

FIG. 12 is a schematic block diagram of an example of a distal end rigid portion of an insertion portion of the ultrasonic endoscope.

FIG. 13 is a flowchart showing processes of a procedure.

### DETAILED DESCRIPTION OF THE PREFERRED EMBODIMENTS

Now, the present invention will be described with reference to a shown embodiment.

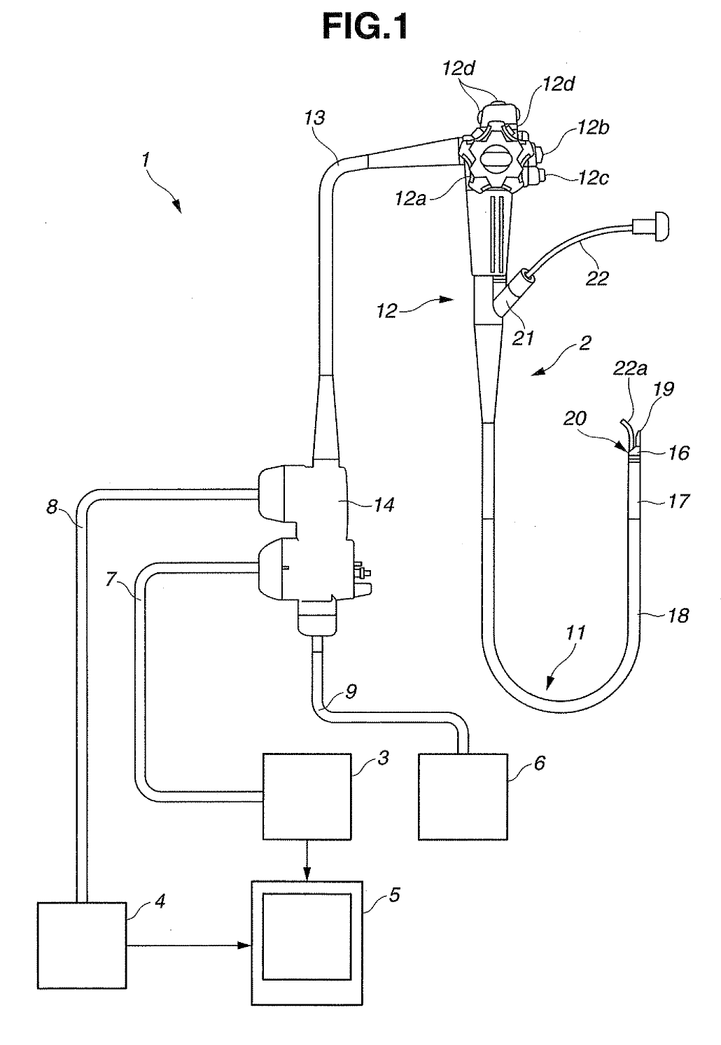

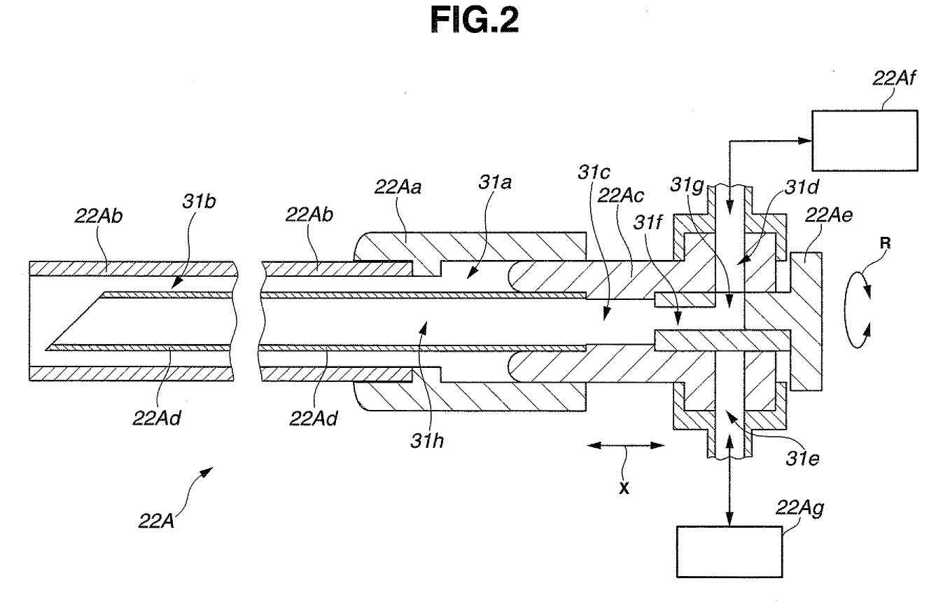

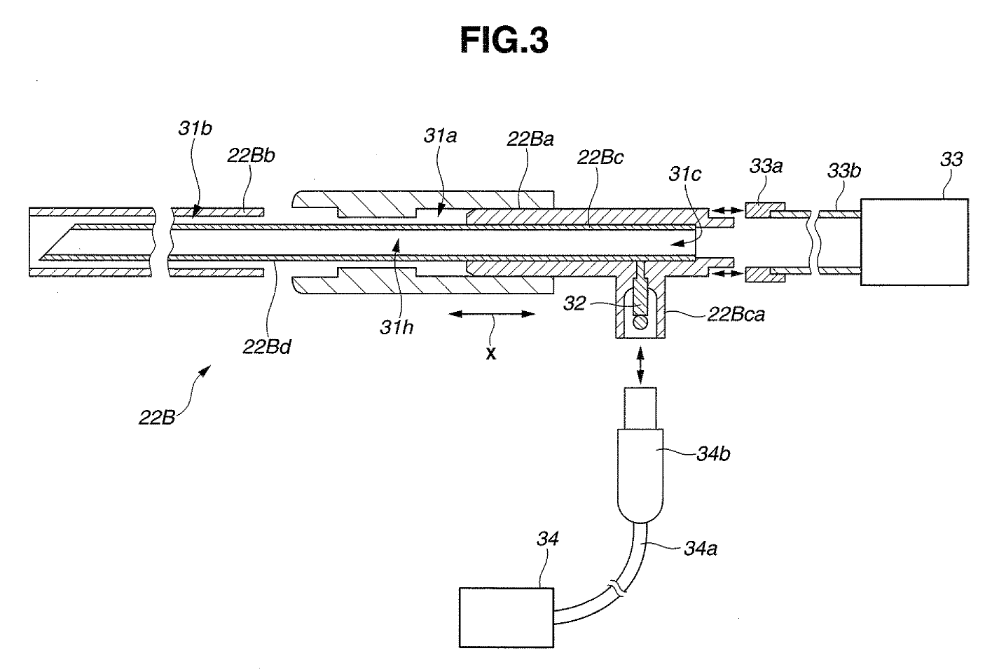

FIG. 1 is a schematic block diagram of the entire configuration of an ultrasonic endoscope system of an embodiment of the present invention. FIG. 2 is a schematic block diagram of an example of a treatment instrument or the like in the ultrasonic endoscope system of The present embodiment. FIG. 3 is a schematic block diagram of another example of a treatment instrument or the like in the ultrasonic endoscope system of the present embodiment.

As shown in FIG. 1, the ultrasonic endoscope system 1 of the present embodiment mainly includes an ultrasonic endoscope 2 including an endoscope observing unit 20 having an endoscope observing function and an ultrasonic observing unit 19 having an ultrasonic observing function, and an endoscope observing apparatus 3 that controls the endoscope observing unit 20 and processes output signals thereof an ultrasonic observing apparatus 4 that controls the ultrasonic observing unit 19 and processes output signals thereof, a display apparatus 5 that receives the signals from the endoscope observing apparatus 3 and the ultrasonic observing apparatus 4 and appropriately displays endoscopic images or ultrasonic tomograms, a light source device 6 that is a light source of an illumination light emitted from a distal end front surface of the endoscope observing unit 20, a video cable 7, an ultrasonic cable 8, and a light source cable 9 and the like.

The ultrasonic endoscope 2 in the ultrasonic endoscope system 1 is, for example, a forward-viewing convex type ultrasonic endoscope in which an observing direction of an image pickup device and a direction of a distal end opening of a treatment instrument channel are placed in substantially parallel to an endoscope inserting axis. Depending on circumstances of use of the system, the endoscope itself does not always require a treatment instrument channel as in the case where an overtube having a treatment instrument channel is mounted to the endoscope and used. Thus, the ultrasonic endoscope 2 applied to the system may be an ultrasonic endoscope having no treatment instrument channel.

The ultrasonic endoscope 2 mainly includes an elongated insertion portion 11 to be inserted into a body cavity, an operation portion 12 connected to a proximal end of the insertion portion 11 for operating the insertion portion 11, a universal cable 13 extending from a side of the operation portion 12, and a connector portion 14 provided at one end of the universal cable 13 and the like.

The insertion portion 11 mainly includes, in order from a distal end side, a distal end rigid portion 16 formed of a rigid member, a bending portion 17 connected to a proximal end of the distal end rigid portion 16 and bendably configured, and a flexible tube 18 having one end connected to a proximal end of the bending portion 17 and the other end connected to a distal end of the operation portion 12, having a small diameter, a long length and flexibility. A treatment instrument channel 41 is formed through the insertion portion 11 from the proximal end to the distal end.

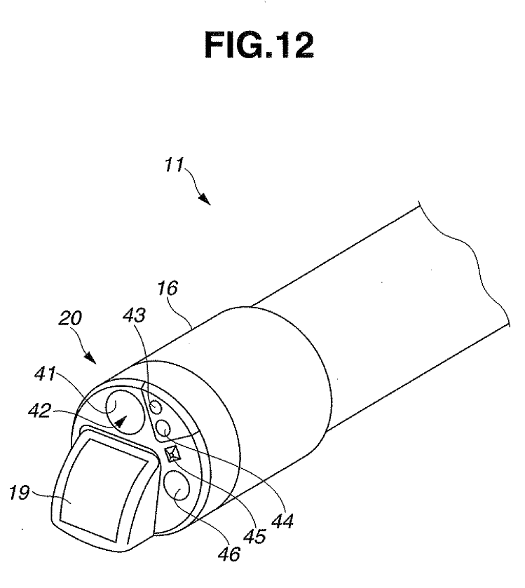

As shown in FIG. 12, the endoscope observing unit 20 and the ultrasonic observing unit 19 are provided at a distal end side of the distal end rigid portion 16.

The ultrasonic observing unit 19 includes a plurality of ultrasonic transducers that transmit and receive ultrasonic waves, arranged to form a ultrasonic scanning surface, and is configured to obtain ultrasonic signals contributing to creating tomograms (ultrasonic tomograms) of the inside of a body cavity wall.

The endoscope observing unit 20 includes an observation optical system member, an illumination optical system member, and an image pickup device, and is configured to obtain image pickup signals contributing to generating image signals for optically picking up images of a body cavity wall surface and displaying endoscopic images for observation.

At the distal end of the distal end rigid portion 16, there are provided an illumination window 43 that constitutes the illumination optical system member, an objective lens 44 that constitutes the observation optical system member, an air/water feeding nozzle 45 that sprays a fluid such as water or air toward the objective lens 44, and a sub-channel 46 for feeding water forward. The sub-channel 46 also has functions for sucking and for inserting the treatment instrument.

The operation portion 12 includes operation members for various operations of the ultrasonic endoscope 2 such as an angle knob 12a that is an operation member for vertically and laterally bending the bending portion 17 in the insertion portion 11, a suction button 12b for a suction operation, an air/water feeding button 12c for feeding air and water, and a plurality of operation members 12d for various operations such as switching displays of the display apparatus 5 or giving freeze instructions or release instructions of display images.

The operation portion 12 is provided with a treatment instrument insertion opening 21 near the distal end thereof in a protruding manner, which is an insertion opening for inserting a treatment instrument or the like 22 of various types through the treatment instrument channel 41 in the insertion portion 11 and introducing the treatment instrument or the like 22 into the body cavity in use of the ultrasonic endoscope 2.

As described above, the universal cable 13 is provided to extend from the side of the operation portion 12, through which a plurality of signal lines that transmit electrical signals or a bundle of optical fiber cables for illumination light are placed. At a distal end portion of the universal cable 13, a connector portion 14 is provided for ensuring connections between the ultrasonic endoscope 2 and the endoscope observing apparatus 3, the ultrasonic observing apparatus 4, and the light source device 6, respectively.

The endoscope observing apparatus 3 is image processing means for driving and controlling the image pickup device in the endoscope observing unit 20 of the ultrasonic endoscope 2, and thus receiving image pickup signals transmitted from the image pickup device to perform various signal processings and generate video signals for endoscope observation images.

The ultrasonic observing apparatus 4 is an ultrasonic image processing device that drives and controls the ultrasonic transducers in the ultrasonic observing unit 19 of the ultrasonic endoscope 2, thus transmits ultrasonic waves of a predetermined frequency toward an object to be observed, receives electrical signals from the ultrasonic transducers, the electrical signals being obtained by receiving ultrasonic waves reflected from the object to be observed, and performs various signal processings to generate video signals for ultrasonic tomograms.

The display apparatus 5 receives the video signals generated by the ultrasonic observing apparatus 4 and the endoscope observing apparatus 3 to switch or simultaneously display corresponding observation images, that is, ultrasonic tomograms or endoscopic images.

The light source device 6 supplies illumination light for illuminating the front of the ultrasonic endoscope 2 via the illumination optical system member provided on a distal end front surface of the endoscope observing unit 20 of the ultrasonic endoscope 2.

The ultrasonic cable 8 is a connecting cable for electrically connecting between the ultrasonic observing apparatus 4 and the ultrasonic endoscope 2.

The video cable 7 is a connecting cable for electrically connecting between the endoscope observing apparatus 3 and the ultrasonic endoscope 2.

The light source cable 9 is an optical fiber cable including a bundle of optical fibers for connecting between the light source device 6 and the ultrasonic endoscope 2, and guiding illumination light from the light source device 6 to the illumination optical system in the endoscope observing unit 20 of the ultrasonic endoscope 2.

The treatment instrument insertion opening 21 provided in the operation portion 12 of the ultrasonic endoscope 2 communicates with the treatment instrument channel 41 formed through the insertion portion 11 from the operation portion 12 to a distal end opening 42 provided in a front surface of the distal end rigid portion 16. Thus, the treatment instrument or the like 22 inserted through the treatment instrument insertion opening 21 passes through the treatment instrument channel 41 and can be protruded from and retracted into the distal end opening 42 of the insertion portion 11.

In addition, through the sub-channel 46 as another channel provided in the distal end rigid portion 16 can be inserted another treatment instrument channel or the like 22 which is different from the treatment instrument or the like 22 inserted through the treatment instrument channel 41.

The treatment instrument or the like 22 inserted through the treatment instrument insertion opening 21 includes, for example, a perforating treatment instrument that is an endoscope needle type treatment instrument having perforating means for perforating a desired region in a luminal organ under observation by the ultrasonic endoscope 2, an insufflator having insufflation means for insufflating the abdominal cavity by puncturing from the inside of the luminal organ before perforating the desired region in the luminal organ, or a marking instrument having marking means for marking the desired region in the luminal organ before insufflation with the insufflator.

Into the treatment instrument insertion opening 21, a surgical treatment instrument 23 for performing surgery of an organ in the abdominal cavity under observation by the ultrasonic endoscope 2, and a suturing instrument 24 that sutures the perforated portion perforated by the perforating treatment instrument under observation by the ultrasonic endoscope 2 are inserted to perform various treatments such as surgery of an organ in the abdominal cavity under observation by the ultrasonic endoscope 2.

The treatment instrument or the like 22 also includes a treatment instrument having two or more functions besides the above described various treatment instruments each having a single function. For example, FIG. 2 shows an example of a treatment instrument having an insufflation function and a marking function by tattooing or the like. FIG. 3 shows an example of a treatment instrument having an insufflation function, and also a marking function and a treatment function by a high frequency needle or the like.

An insufflator 22A in FIG. 2 has a slide portion 22Ac that slides in a long axis direction with respect to a main body 22Aa, a needle tube 22Ad connected to the slide portion 22Ac is moved in the same direction to have a puncturing function, and a proximal end of the slide portion 22Ac is bifurcated and has a switching mechanism, for example, in the form of a three-way stopcock. A marker supplier 22Af is connected to one of the bifurcated ends and a gas supplier 22Ag is connected to the other. This allows selective switching between the marking function and the insufflation function.

Specifically, the insufflator 22A includes the substantially cylindrical main body 22Aa, a flexible elongated tubular sheath 22Ab connected one end (a distal end) of the main body 22Aa, a tubular slide portion 22Ac provided slidably in the direction of arrow X in the main body 22Aa from the other end thereof the needle tube 22Ad connected to one end (a distal end) of the slide portion 22Ac and formed into a flexible elongated tubular shape and a needle shape having an acute distal end, a switching operation member 22Ae mounted to the other end of the slide portion 22Ac and provided rotatably in the direction of arrow R within a predetermined rotation range, a marker supplier 22Af connected to the slide portion 22Ac via a connector and a connecting tube for supplying a marking member such as tattoo, and a gas supplier 22Ag connected to the slide portion 22Ac via a connector and a connecting tube for supplying gas such as carbon dioxide (CO₂) and so forth.

In a side surface at the other end of the slide portion 22Ac, two side ducts 31d and 31e are provided that circumferentially face each other. The side ducts 31d and 31e are connected to a hollow portion 31c in the slide portion 22Ac.

The switching operation member 22Ae has a hollow portion 31f formed from one end to a middle portion in the long axis direction. In a side surface of the switching operation member 22Ae, a side duct 31g connected to the hollow portion 31f is provided. The side duct 31g is formed in a position facing the side ducts 31d and 31e when the switching operation member 22Ae is mounted to a predetermined region at the other end of the slide portion 22Ac.

Thus, if the switching operation member 22Ae is rotated in the direction of arrow R in FIG. 2 with the switching operation member 22Ae being rotatably mounted to the other end of the slide portion 22Ac, in a predetermined position, the side duct 31g in the switching operation member 22Ae communicates with the side duct 31d in the slide portion 22Ac, while the side duct 31e in the slide portion 22Ac is blocked. Similarly, in another predetermined position, the side duct 31g in the switching operation member 22Ae communicates with the side duct 31e in the slide portion 22Ac, while the side duct 31d in the slide portion 22Ac is blocked.

As described above, the slide portion 22Ac is slidably mounted to the other end of the main body 22Aa. The needle tube 22Ad is connected to one end of the slide portion 22Ac. Thus, the hollow portion 31c in the slide portion 22Ac communicates with the hollow portion 31h in the needle tube 22Ad.

As described above, the sheath 22Ab is connected to one end of the main body 22Aa, and thus the hollow portion 31a in the main body 22Aa communicates with the hollow portion 31b in the sheath 22Ab. In this state, the needle tube 22Ad is placed through the hollow portion 31b in the sheath 22Ab.

The marker supplier 22Af is connected to the side duct 31d in the slide portion 22Ac via a connector and a connecting tube.

Similarly, the gas supplier 22Ag is connected to the side duct 31e in the slide portion 22Ac via a connector and a connecting tube.

In the insufflator 22A thus configured, the slide portion 22Ac is slid in the direction of arrow X with respect to the main body 22Aa, and thus the needle tube 22Ad is moved in the sheath 22Ab in the same direction. Thus, the needle tube 22Ad is protruded from and retracted into the distal end opening 42 in the sheath 22Ab.

The switching operation member 22Ae is rotated in the direction of arrow R and placed in a predetermined position to form a marker duct from the side duct 31d to which the marker supplier 22Af is connected, via the hollow portions 31f, 31c and 31h, Thus, a marking member such as tattoo supplied from the marker supplier 22Af can be supplied to the distal end opening 42 of the needle tube 22Ad to implement a marking function (the state in FIG. 2).

Similarly, the switching operation member 22Ae is rotated in the direction of arrow R and placed in another predetermined position to form a gas duct from the side duct 31e to which the gas supplier 22Ag is connected, via the hollow portions 31f, 31c and 3h. Thus, gas supplied from the gas supplier 22Ag can be supplied to the distal end opening 42 of the needle tube 22Ad to implement the insufflation function.

On the other hand, an insufflator 22B in FIG. 3 has a slide portion 22Bc that slides in a long axis direction with respect to a main body 22Ba, a needle tube 22Bd connected to the slide portion 22Bc is moved in the same direction to have a puncturing function, and to a proximal end of the slide portion 22Bc, an insufflator 33 that implements an insufflation function and a high frequency wave generator 34 that implements a marking function and a treatment function are connected.

Specifically, the insufflator 22B includes the substantially cylindrical main body 22Ba, a flexible elongated tubular sheath 22Bb connected to one end (a distal end) of the main body 22Ba, a tubular slide portion 22Bc provided slidably in the direction of arrow X in the main body 22Ba from the other end thereof the needle tube 22Bd connected to one end (a distal end) of the slide portion 22Bc, formed into a flexible elongated tubular shape and a needle shape having an acute distal end, and formed of a conductive member, a gas supplier 33 connected to the other end of the slide portion 22Bc via a connector 33a and a connecting tube 33b for supplying gas, and the high frequency wave generator 34 connected to the slide portion 22Bc via a connector 34b and a power supply cord 34a.

In a predetermined region on a side surface at a proximal end of the slide portion 22Bc, a connector portion 22Bca protruding outward is provided. In the connector portion 22Bca, a connector 34b of the power supply cord 34a is removably provided.

In the connector portion 22Bca, an electrode 32 formed of a conductive member is provided. One end of the electrode 32 is connected to the needle tube 22Bb secured to an inner surface of the slide portion 22Bc. The other end of the electrode 32 is brought into contact with a contact point of the connector 34b of the power supply cord 34a when the connector 34b fits the connector portion 22Bca. Thus, high frequency waves from the high frequency wave generator 34 are transmitted to the distal end portion of the needle tube 22Bd through the power supply cord 34a, the connector 34b, and the electrode 32, and for example, the marking function and the treatment function such as perforation can be implemented.

As described above, the slide portion 22Bc is slidably mounted to the other end of the main body 22Ba. The needle tube 22Bd is connected to one end of the slide portion 22Bc. Thus, a hollow portion 31c in the slide portion 22Bc communicates with a hollow portion 31h in the needle tube 22Bd.

As described above, the sheath 22Bb is connected to one end of the main body 22Ba, and thus the hollow portion 31a in the main body 22Ba communicates with the hollow portion 31b in the sheath 22Bb. In this state, the needle tube 22Bd is placed through the hollow portion 31b in the sheath 22Bb.

To the other end of the slide portion 22Bc, the gas supplier 33 is connected via the connector 33a and the connecting tube 33b. Thus, the gas supplier 33 communicates with the hollow portion 31c in the slide portion 22Bc via the connecting tube 33b and the connector 33a.

In the insufflator 22B thus configured, the slide portion 22Bc is slid in the direction of arrow X with respect to the main body 22Ba, and thus the needle tube 22Bd is moved in the sheath 22B in the same direction. Thus, the needle tube 22Bd is protruded from and retracted into the distal end opening 42 in the sheath 22Bb.

The gas supplier 33 is operated to spray gas from the gas supplier 33 forward from the distal end portion of the needle tube 22Bd through a channel constituted by the connecting tube 33b and the connector 33a, the hollow portion 31c in the slide portion 22Bc, and the hollow portion 31h in the needle tube 22Bd.

Further, the high frequency waves generated by driving and controlling the high frequency wave generator 34 are transmitted to the distal end of the needle through the electrode 32 of the connector 22Bca and the needle tube 22Bd. Thus, the high frequency wave generator 34 is driven and controlled with the needle tube 22Bd being protruded from the sheath 22Bb to allow marking treatment or the like at the distal end portion of the needle tube 22Bd, and the marking function and the treatment function are implemented.

Thus, the treatment instruments in FIGS. 2 and 3 are configured to have the making function for marking a desired region in the luminal organ and also have the insufflation function for performing insufflation by puncturing the luminal organ and causing the distal end of the needle to reach the abdominal cavity, by forming the needle tube placed through the sheath to have the hollow structure and the needle-shaped distal end. In the example in FIG. 3, the treatment instrument further has a high frequency treatment function. Thus, the treatment instrument having both the marking function and the insufflation function (also the treatment function) is used to allow omitting replacement of treatment instruments corresponding to procedure steps into the treatment instrument channel in the endoscope insertion portion, thereby simplifying treatment steps to reduce treatment time.

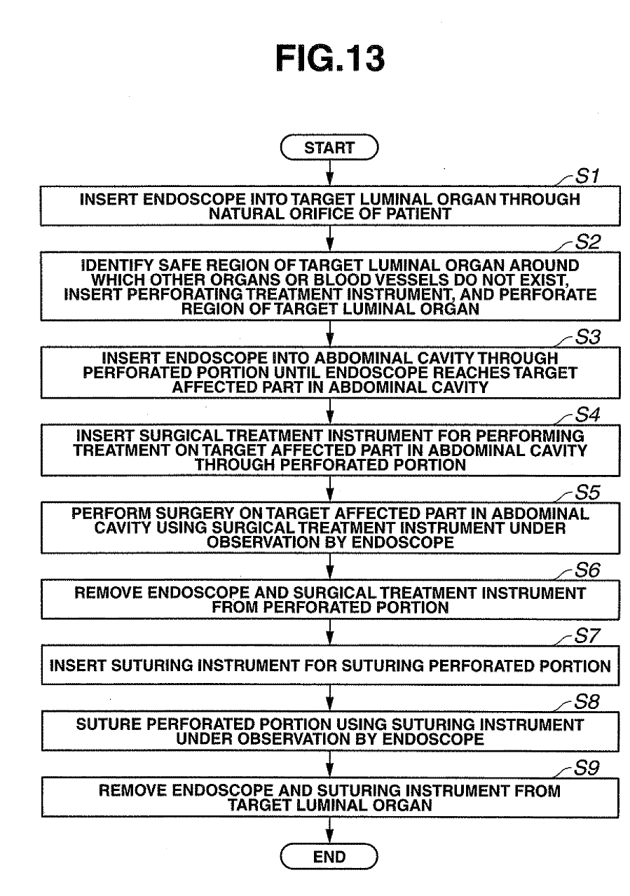

Next, the procedure according to the present invention will be described with reference to FIG. 13. FIG. 13 is a flowchart showing processes of the procedure.

As shown in FIG. 13, in the procedure of the present invention, first in step S1, an endoscope is inserted into a target luminal organ through a natural orifice of a subject.

Next, in step S2, a region of the target luminal organ, which is a safe region without other organs or blood vessels, is identified under observation by the endoscope, and the region of the target luminal organ is perforated using the perforating treatment instrument.

Next, in step S3, the endoscope is inserted into the abdominal cavity through the perforated portion which is the region perforated in the step S2 until the endoscope reaches a target affected part in the abdominal cavity.

Next, in step S4, the surgical treatment instrument for performing treatment on the affected part in the abdominal cavity is inserted through the perforated portion. As the surgical treatment instrument, a type which is inserted through the channel of the endoscope and a type which is covered on the endoscope, so-called overtube type, can be used.

Then, in step S5, surgery of the target affected part in the abdominal cavity is performed using the surgical treatment instrument under observation by the endoscope. After that, in step S6, the endoscope and the surgical treatment instrument are removed from the perforated portion.

Next, in step S7, the suturing instrument for suturing the perforated portion is inserted, and in step S8, the perforated portion is sutured using the suturing instrument under observation by the endoscope. Then, in step S9, the endoscope and the suturing instrument are removed from the target luminal organ and the procedure ends.

The procedure of the present invention is characterized in that, in at least one step of the above-described steps S1 to S9, observation using an ultrasonic tomogram obtained by the ultrasonic endoscope 2 or observation using an ultrasonic tomogram obtained by the ultrasonic probe inserted through the treatment instrument channel of the endoscope is adopted instead of observation using an optical image picked up by the endoscope.

In other words, in the present invention, in at least one step of the above-described steps S2, S3, S5, and S8, observation using the ultrasonic tomogram obtained by the ultrasonic endoscope 2 or the ultrasonic probe is performed. Needless to say, the present invention includes a case in which the ultrasonic endoscope 2 or the ultrasonic probe is used in all of the steps S1 to S9.

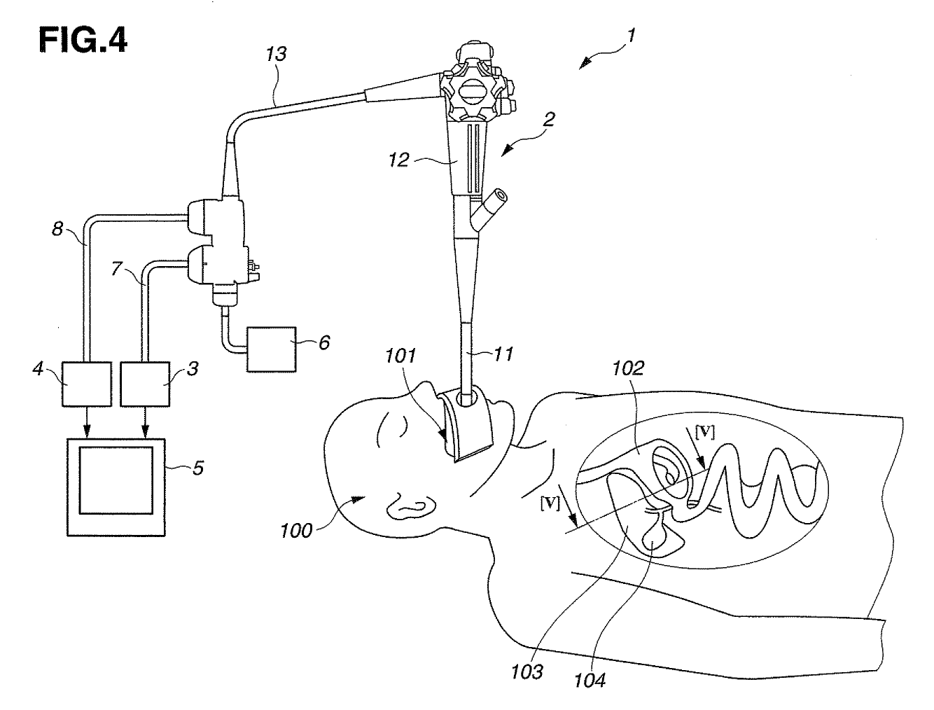

Next, the procedure performed using the ultrasonic endoscope system 1 of the present embodiment will be described with reference to FIGS. 4 to 11. FIGS. 4 to 11 illustrate a process of the procedure, and conceptually show, for example, an arrangement of various organs and blood vessels in the body cavity.

Surgery by the NOTES procedure performed using the ultrasonic endoscope system 1 of the present embodiment includes cholecystectomy.

The outline of the surgery by the NOTES procedure is that an opening is formed in an alimentary tract wall while observing images from an endoscope introduced into the alimentary tract through a natural orifice, the endoscope is introduced into an abdominal cavity through the opening to perform observation and treatment, and the opening is sutured after the treatment.

Specifically, the following steps axe performed in order.

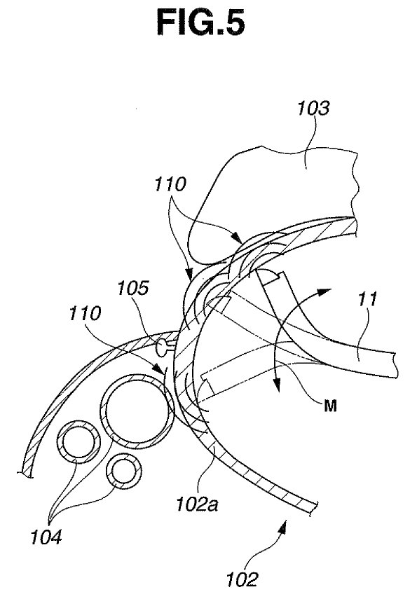

(1) First, various instruments in the ultrasonic endoscope system 1 are energized to make the system available. In this state, while observing endoscopic images displayed on the display apparatus 5, the insertion portion 11 of the ultrasonic endoscope 2 is inserted through a natural orifice, for example, an oral cavity 101 of a subject 100 into a target luminal organ, for example, a stomach 102 to be formed with an opening in a wall thereof. For the target luminal organ, the endoscope can be inserted through the opening in the wall thereof into the abdominal cavity in a normal process. The insertion operation of the ultrasonic endoscope 2 in this case is similar to that in a flexible endoscopy generally performed (see FIG. 4). FIG. 4 shows the stomach 102 in a partially broken state.
(2) With the distal end portion of the ultrasonic endoscope 2 reaching inside the target luminal organ (stomach 102), a state in the abdominal cavity outside the luminal organ is observed through a stomach wall 102a of the stomach 102 by an ultrasonic observing function of the ultrasonic endoscope 2 besides the observation of the inside of the stomach 102 by an endoscope function of the ultrasonic endoscope 2. At this time, the operation member of the operation portion 12 is operated to perform a bending operation of the distal end portion of the insertion portion 11 (see FIG. 5). The arrow M in FIG. 5 shows that the distal end portion is moved by the bending operation.

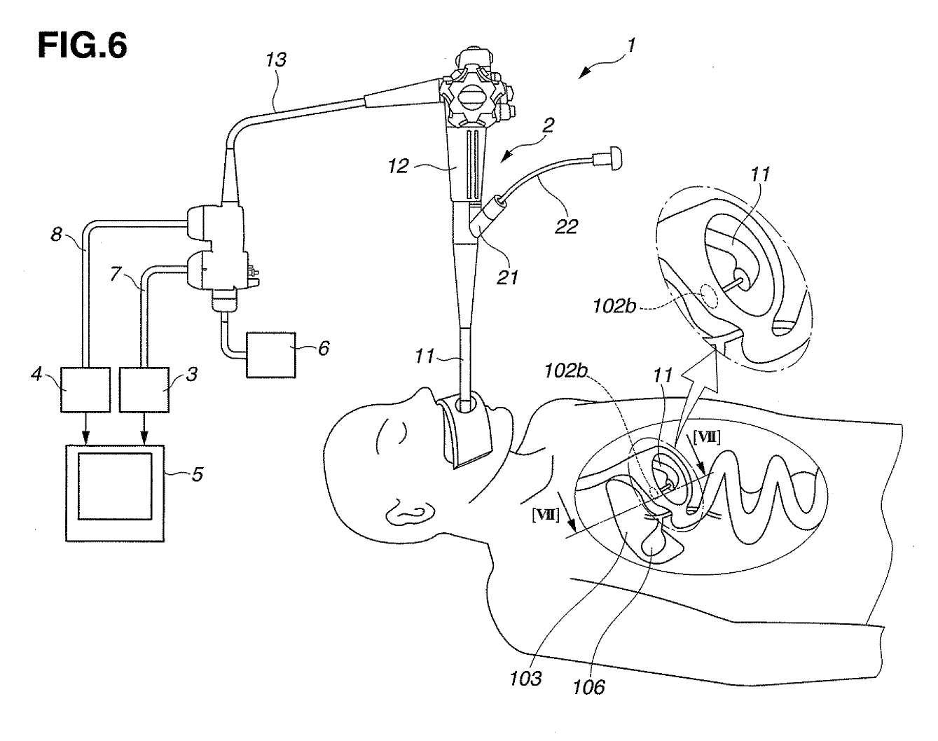

The state in the abdominal cavity outside the stomach 102 is unclear only by observing the endoscopic images by the endoscope function using the ultrasonic endoscope 2 from the inside of the stomach 102. As shown in an enlarged view of essential portions in FIG. 5, there are a liver 103, an intestine 104, a gallbladder 106 (not shown in FIG. 5, see FIGS. 4 and 6 or the like) in the abdominal cavity outside and near the stomach 102. There are also arteries 105 or the like. In FIG. 5, reference numeral 110 conceptually shows a state of emission of ultrasonic waves from the ultrasonic endoscope 2.

In the step (2), the state in the abdominal cavity outside the stomach 102 is observed through the stomach wall 102a by the ultrasonic observing function of the ultrasonic endoscope 2 to identify a region without other organs or blood vessels.

Specifically, in the NOTES procedure, for the distal end portion (the treatment instrument protruded from the distal end portion) of the insertion portion 11 of the ultrasonic endoscope 2 to reach the target organ outside the stomach 102, the stomach wall 102a is first punctured. The step (2) is for identifying a position for puncturing.

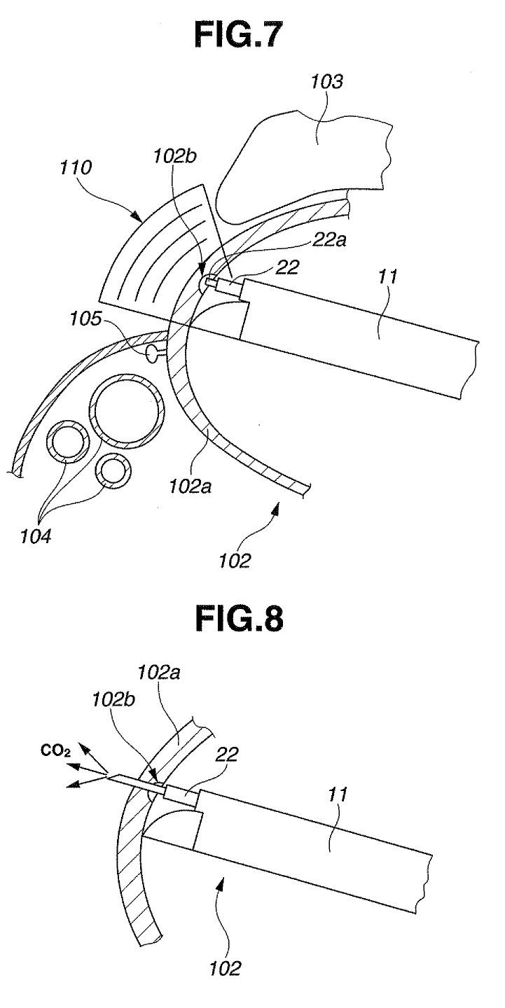

To identify a position for making an dissection in the stomach wall 102a with safety and reliability without damaging other organs or blood vessels outside the stomach wall 102a (in the abdominal cavity), the arrangement of other organs or blood vessels in the abdominal cavity is checked on the ultrasonic tomograms by the ultrasonic observing function of the ultrasonic endoscope 2, and the position for puncturing in the stomach wall 102a is scanned and identified. Then, a marking 102b (see FIGS. 6 and 7) is applied to the identified region by high frequency waves or tattooing.

To apply the marking 102b to the position for puncturing in the stomach wall 102a identified in the above described process (see FIG. 5), a marking instrument (for example, a single marking treatment instrument or the insufflators 22A and 22B in FIGS. 2 and 3) having marking means is first inserted into the treatment instrument insertion opening 21 in the ultrasonic endoscope 2, and placed through the insertion portion 11 through the treatment instrument channel 41 (see FIG. 6).

Then, the distal end portion 22a of the marking instrument is placed near the identified region, and a predetermined operation is performed to perform predetermined marking treatment (see FIG. 7). Then, the marking instrument is removed from the treatment instrument channel 41 and the treatment instrument insertion opening 21 as required. The removal operation is not required in the treatment instrument having both the marking function and the insufflation function as shown in FIGS. 2 and 3 or the like.

(3) Then, the stomach wall 102a is punctured to perform insufflation treatment for insufflating the abdominal cavity. The insufflation treatment is performed by an insufflator. Thus, a predetermined insufflator (for example, see FIGS. 2 and 3 or the like) is first set in the ultrasonic endoscope 2.

Specifically, the insufflator is inserted into the treatment instrument insertion opening 21 and placed through the treatment instrument channel 41. Then, a distal end portion of the insufflator is protruded through the distal end opening 42 of the treatment instrument channel 41 in the ultrasonic endoscope 2, and the region with the marking 102b identified in the step (2) or the region with safety ensured is punctured with the distal end portion for insufflation under observation of the endoscopic images from the ultrasonic endoscope 2 and also under ultrasonic images for further safety (see FIG. 8).

Then, the insufflator is removed from the treatment instrument channel 41 and the treatment instrument insertion opening 21 as required. The removal operation is not required when the insufflator 22B in FIG. 3 is used, that is, the treatment instrument that can perform marking and treatment with high frequency waves and also perform insufflation treatment.

(4) Next, a perforating treatment instrument for making a dissection in the stomach wall 102a, for example, a high frequency snare (not shown) is set in the ultrasonic endoscope 2.

Specifically, the perforating treatment instrument is inserted into the treatment instrument insertion opening 21 and placed through the treatment instrument channel 41. Then, a distal end portion of the perforating treatment instrument is protruded through the distal end opening 42 in the ultrasonic endoscope 2, and placed near the region with the marking 102b identified in the step (2), and the identified region 102b is perforated with the perforating treatment instrument under observation of endoscopic images and ultrasonic tomograms from the ultrasonic endoscope 2. Then, the perforating treatment instrument is removed from the treatment instrument channel 41 and the treatment instrument insertion opening 21 as required. The removal operation is not required when the insufflator 22B in FIG. 3 is used, that is, the treatment instrument that can perform marking and treatment with high frequency waves and also perform insufflation treatment.

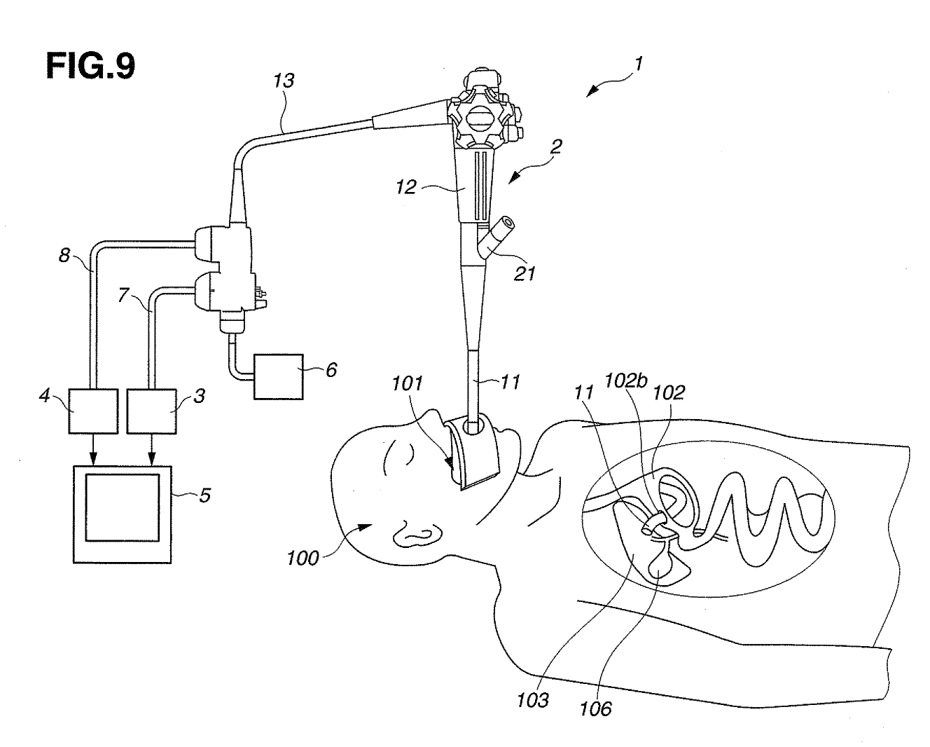

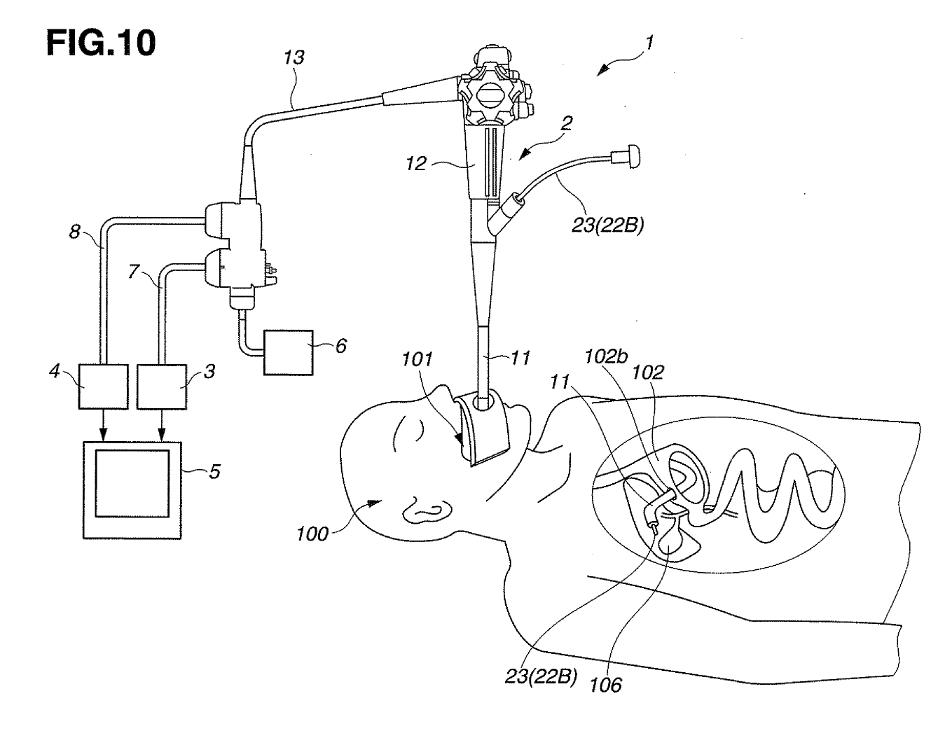

(5) After the distal end portion of the ultrasonic endoscope 2 is inserted from the identified region 102b in the stomach wall 102a perforated in the step (4) to puncture the stomach wall 102a, the target organ in the abdominal cavity is observed on the endoscopic images from the ultrasonic endoscope 2 (see FIG. 9). FIG. 9 shows the state where the perforating treatment instrument is removed from the treatment instrument channel 41 and the treatment instrument insertion opening 21, while when the treatment instrument in FIG. 3 is used, the treatment instrument is placed through the treatment instrument channel 41 and the treatment instrument insertion opening 21 also in this state (the latter case is not shown).
(6) Next, surgery of the target organ in the abdominal cavity (for example, the gallbladder 106 in the case of cholecystectomy) under observation of endoscopic images and ultrasonic tomograms from the ultrasonic endoscope 2. Thus, a surgical treatment instrument 23 (22B) is first inserted through the treatment instrument insertion opening 21 in the ultrasonic endoscope 2, and the surgical treatment instrument 23 (22B) is placed through the treatment instrument channel 41 (see FIG. 10 or the like).

The surgery herein is performed under observation of the endoscopic images and the ultrasonic tomograms from the ultrasonic endoscope 2, and thus the procedure can be performed without damaging regions near the target region in the abdominal cavity or regions other than the surgery target such as blood vessels in organs that are not directly visible or other organs. When the surgery is completed, the surgical treatment instrument is removed from the treatment instrument channel 41 and the treatment instrument insertion opening 21 in the ultrasonic endoscope 2. Further, the distal end portion of the ultrasonic endoscope 2 inserted into the perforated portion (102b) is removed from the abdominal cavity. At this time, the distal end portion of the ultrasonic endoscope 2 is retained in the stomach 102.

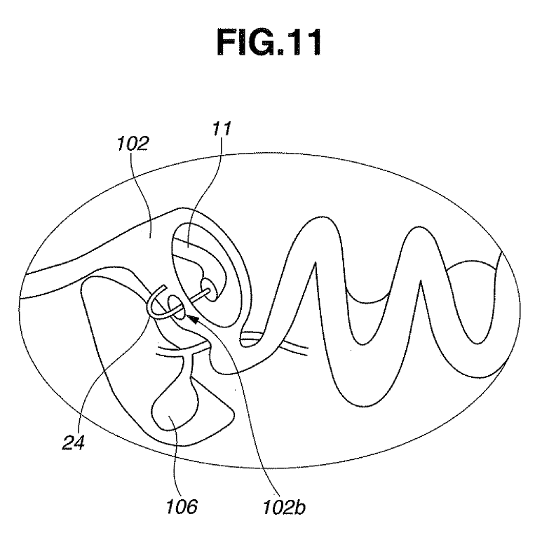

(7) Then, the perforated portion 102b in the stomach wall 102a perforated in the step (4) is sutured under observation of endoscopic images from the ultrasonic endoscope 2 retained in the stomach 102. Thus, the suturing instrument 24 is inserted through the treatment instrument insertion opening 21 in the ultrasonic endoscope 2, and placed through the treatment instrument channel 41 (not shown, see FIG. 11). Then, suturing treatment of the perforated portion 102b is performed in a predetermined process using the suturing instrument 24, for example, a clip protruded from the distal end of the ultrasonic endoscope 2. When the suturing treatment is completed, the suturing instrument 24 is removed from the treatment instrument channel 41 and the treatment instrument insertion opening 21 in the ultrasonic endoscope 2. Then, the ultrasonic endoscope 2 is removed from the luminal organ (stomach 102).

As described above, according to the first embodiment, in the surgery with the NOTES procedure, that is, the procedure for the surgery of the organ in the abdominal cavity from the luminal organ under observation by the ultrasonic endoscope 2, the position without other organs or blood vessels outside the luminal organ is identified on the ultrasonic tomograms from the ultrasonic endoscope 2, and the identified position is perforated. This prevents damaging undesired regions such as other organs or blood vessels other than the organ in the abdominal cavity to be subjected to the surgery in the case of puncturing with the needle tube from the luminal organ for insufflating the abdominal cavity or perforation by making an dissection in the inner wall surface (stomach wall or the like) of the luminal organ.

All the procedure steps can be performed under observation of observation images from the ultrasonic endoscope 2, and thus a step of replacement of the endoscope itself can be omitted. This allows quick and efficient treatment while ensuring safety.

Specifically, for example, a large cystic artery runs in the gallbladder, and in the cholecystectomy, a region with the cystic artery can be identified under observation of the ultrasonic tomograms from the ultrasonic endoscope 2. Thus, hemostasis of the cystic artery can be performed by means such as a clip, which ensures more safety.

There are large variations among individuals in positions of a gallbladder duct and an accessory hepatic duct in the abdominal cavity, and as means for preventing damaging the organ and the duct, performing the procedure under observation using the ultrasonic observing function of the ultrasonic endoscope 2 in surgery of the gallbladder of the lever and applying the system and the procedure of the present invention is significantly useful.

Note that, in the above-described cholecystectomy, description is made assuming that the insufflation treatment and the marking treatment are performed. However, the insufflation treatment and the marking treatment are arbitrarily performed as needed. That is, the procedure of the present invention can include a case in which at least one of the insufflation treatment and the marking treatment is not performed.

For example, the marking treatment can be omitted, if the region where other organs or blood vessels are not damaged is checked under the observation using the ultrasonic tomogram from the ultrasonic endoscope 2, and the insufflation treatment and the perforating treatment are performed as-is while checking safety in real-time using the ultrasonic tomogram from the ultrasonic endoscope 2. As a result, the procedure can be performed more rapidly and effectively.

The present invention is not limited to the above described embodiment, but various modifications and applications can be made without departing from the gist of the invention. Further, the present embodiment includes inventions of various stages, and various inventions can be obtained depending on combinations of a disclosed plurality of constituent elements.

What is claimed is:

1. A procedure comprising the steps of:
inserting an endoscope into a target luminal organ through a natural orifice of a subject;
checking and identifying a region without other organs or blood vessels under observation by the endoscope;
inserting a perforating treatment instrument for perforating the target luminal organ under observation by the endoscope;
perforating the region where safety in an abdominal cavity outside the target luminal organ is checked in the checking step, using the perforating treatment instrument;
inserting the endoscope into the abdominal cavity through the perforated portion;
inserting a surgical treatment instrument for performing treatment of an organ in the abdominal cavity through the perforated portion;
performing surgery under observation by the endoscope;
removing the endoscope and the surgical treatment instrument from the perforated portion;
inserting a suturing instrument for suturing the perforated portion under observation by the endoscope; and
suturing the perforated portion using the suturing instrument under observation by the endoscope, wherein the endoscope is replaced by an ultrasonic endoscope in at least one of the steps.

2. A procedure comprising the steps of:
inserting an endoscope into a target luminal organ through a natural orifice of a subject;
checking and identifying a region without other organs or blood vessels under observation by the endoscope;
inserting an insufflation treatment instrument for insufflating an abdominal cavity around the target luminal organ;
insufflating the abdominal cavity using the insufflation treatment instrument in the region where safety in the abdominal cavity outside the target luminal organ is checked in the checking step;
inserting a perforating treatment instrument for perforating the target luminal organ under observation by the endoscope;
perforating the region where safety in the abdominal cavity outside the target luminal organ is checked in the checking step, using the perforating treatment instrument;
inserting the endoscope into the abdominal cavity through the perforated portion;
inserting a surgical treatment instrument for performing treatment of an organ in the abdominal cavity through the perforated portion;
performing surgery under observation by the endoscope;
removing the endoscope and the surgical treatment instrument from the perforated portion;
inserting a suturing instrument for suturing the perforated portion under observation by the endoscope; and
suturing the perforated portion using the suturing instrument under observation by the endoscope, wherein the endoscope is replaced by an ultrasonic endoscope in at least one of the steps.

3. A procedure comprising the steps of:
inserting an endoscope into a target luminal organ through a natural orifice of a subject;
checking and identifying a region without other organs or blood vessels under observation by the endoscope;
marking the region identified in the checking step using a marker;
inserting an insufflation treatment instrument for insufflating an abdominal cavity around the target luminal organ;
insufflating the abdominal cavity using the insufflation treatment instrument in the region where safety in the abdominal cavity outside the target luminal organ is checked in the checking step;
inserting a perforating treatment instrument for perforating the target luminal organ under observation by the endoscope;
perforating the region where safety in the abdominal cavity outside the target luminal organ is checked in the checking step, using the perforating treatment instrument;
inserting the endoscope into the abdominal cavity through the perforated portion;
inserting a surgical treatment instrument for performing treatment of an organ in the abdominal cavity through the perforated portion;
performing surgery under observation by the endoscope;
removing the endoscope and the surgical treatment instrument from the perforated portion;
inserting a suturing instrument for suturing the perforated portion under observation by the endoscope; and
suturing the perforated portion using the suturing instrument under observation by the endoscope, wherein the endoscope is replaced by an ultrasonic endoscope in at least one of the steps.

4. The procedure according to claim 1, wherein the ultrasonic endoscope is replaced by an ultrasonic probe placed through a treatment instrument channel in the endoscope.

5. The procedure according to claim 2, wherein the ultrasonic endoscope is replaced by an ultrasonic probe placed through a treatment instrument channel in the endoscope.

6. The procedure according to claim 3, wherein the ultrasonic endoscope is replaced by an ultrasonic probe placed through a treatment instrument channel in the endoscope.

7. A procedure comprising the steps of:
inserting an ultrasonic endoscope into a target luminal organ through a natural orifice of a subject;
observing the outside of the target luminal organ using the ultrasonic endoscope and checking and identifying a region without other organs or blood vessels;
marking the region identified in the checking step using a marker;
inserting an insufflation treatment instrument for insufflating the region outside the target luminal organ under observation by the ultrasonic endoscope;
insufflating an abdominal cavity using the insufflation treatment instrument under observation by the ultrasonic endoscope;
inserting a perforating treatment instrument for perforating the target luminal organ under observation by the ultrasonic endoscope;
perforating the region where safety in the abdominal cavity outside the target luminal organ is checked in the checking step, using the perforating treatment instrument;
inserting the ultrasonic endoscope through the perforated portion;
inserting a surgical treatment instrument for performing surgery of an organ in the abdominal cavity through the perforated portion;
performing surgery under observation by the ultrasonic endoscope;
removing the ultrasonic endoscope and the surgical treatment instrument from the perforated portion;
inserting a suturing instrument for suturing the perforated portion under observation by the ultrasonic endoscope; and
suturing the perforated portion using the suturing instrument under observation by the ultrasonic endoscope.
